(19) United States
(12) Patent Application Publication
(10) Pub. No.: US 2025/0152944 A1
(43) Pub. Date: May 15, 2025

(54) SYSTEMS AND METHODS TO TITRATE SCS DOSING THROUGH AUTONOMIC MEASURES

(71) Applicant: Boston Scientific Neuromodulation Corporation, Valencia, CA (US)

(72) Inventors: Lisa Denise Moore, Glendale, CA (US); Tianhe Zhang, Studio City, CA (US); Que T. Doan, West Hills, CA (US)

(73) Assignee: Boston Scientific Neuromodulation Corporation, Valencia, CA (US)

(21) Appl. No.: 18/945,383

(22) Filed: Nov. 12, 2024

Related U.S. Application Data
(60) Provisional application No. 63/598,360, filed on Nov. 13, 2023.

(51) Int. Cl. A61N 1/36

(52) U.S. Cl. CPC A61N 1/36139; A61N 1/36062; A61N 1/36071

(57) ABSTRACT

Methods and systems for delivering a therapy and observing one or more metrics of autonomic function. Spinal cord stimulation may be initiated, and patient response monitored using one or more metrics of autonomic function, including heart rate, heart rate variability, or other markers. Therapy intensity may be increased or decreased, or therapy may be changed to use a different program, or therapy may be ceased, depending on changes in the observed metrics of autonomic function.

### CROSS-REFERENCE TO RELATED APPLICATIONS

This application claims the benefit of U.S. Provisional Patent Application Ser. No. 63/598,360, filed Nov. 13, 2023, which is incorporated herein by reference.

### BACKGROUND

Spinal cord stimulation (SCS) is an accepted form of pain therapy. Management of SCS dosing, that is, the determination of how “much” SCS a patient is to receive is often left to the patient's discretion, using a patient remote control (RC) that allows the patient to turn therapy on and off, and to adjustment output parameters within boundaries set by a physician, such as to increase or decrease the amplitude of stimulation. Dosing may be understood as more broadly encompassing the amount of charge delivered over time, including factors such as frequency, pulse width, and amplitude. The use of sensing in SCS has, to date, been limited to, for example, using impedance or field propagation measurements to determine relative positioning of leads in the patient's spinal column, or sensing very short-term neural responses to pulsed stimulation such as evoked compound action potentials. New and alternative uses of sensed data, particularly to aid in management of SCS dosing, are desired.

### Overview

The present inventors have recognized, among other things, that a problem to be solved is the need for new and/or alternative SCS dosing tools. Metrics of autonomic function may be used to determine the appropriateness of existing SCS configuration and utilization in some examples.

An illustrative and non-limiting example takes the form of a an implantable medical device system comprising: a lead adapted for placement in the spinal column of a patient; a sensing circuitry configured to sense a metric of the patient's autonomic function; a pulse generator configured to couple with the lead and containing stimulation circuitry configured to issue pulses via the lead, and control circuitry configured to perform the following: issue pulses to the patient in accordance with a first treatment program; determine, from the sensing circuitry, whether the metric of the patient's autonomic function indicates a risk of overregulation or overmodulation (overregulation in the following examples); and in response to a determination that the metric of the patient's autonomic function indicates a risk of overregulation, reducing the risk of overregulation.

Additionally or alternatively, the control circuitry is configured to determine, from the sensing circuitry, whether the metric of the patient's autonomic function indicates a risk of overregulation by tracking a trend the metric.

Additionally or alternatively, the control circuitry is configured to determine, from the sensing circuitry, whether the metric of the patient's autonomic function indicates a risk of overregulation by: detecting, using the sensing circuitry, heart beats of the patient; determining, from the detected heart beats, a cardiac rate of the patient; and comparing the cardiac rate or a trend of the cardiac rate to a threshold.

Additionally or alternatively, the control circuitry is configured to determine, from the sensing circuitry, whether the metric of the patient's autonomic function indicates a risk of overregulation by: detecting, using the sensing circuitry, heart beats of the patient; determining, from the detected heart beats, a heart rate variability of the patient; and comparing the heart rate variability or a trend of the heart rate variability to a threshold.

Additionally or alternatively, the control circuitry is configured to determine, from the sensing circuitry, whether the metric of the patient's autonomic function indicates a risk of overregulation by: detecting, using the sensing circuitry, a respiration rate of the patient; comparing the respiration rate, or a trend of the respiration rate, to a threshold.

Additionally or alternatively, the control circuitry is configured to determine, from the sensing circuitry, whether the metric of the patient's autonomic function indicates a risk of overregulation by: detecting, using the sensing circuitry, heart beats of the patient; analyzing the heart beats of the patient to yield a first estimate of the patient's autonomic state; obtaining data from a second device to yield a second estimate of the patient's autonomic state, wherein the second device provides an indication of blood pressure.

Additionally or alternatively, the control circuitry is configured to determine, from the sensing circuitry, whether the metric of the patient's autonomic function indicates a risk of overregulation by: detecting, using the sensing circuitry, heart beats of the patient; analyzing the heart beats of the patient to yield a first estimate of the patient's autonomic state; obtaining data from a second device to yield a second estimate of the patient's autonomic state, wherein the second device provides an indication of galvanic skin resistance of the patient.

Additionally or alternatively, the control circuitry is configured to determine, from the sensing circuitry, whether the metric of the patient's autonomic function indicates a risk of overregulation by determining a first autonomic function marker and a second autonomic function marker each selected from heart rate, heart rate variability, blood pressure, respiration rate, galvanic skin response, bladder, bowel or sexual function, posture and myopotentials, and using the first autonomic function marker and the second autonomic function marker to estimate the metric.

Additionally or alternatively, the control circuitry is configured to determine, from the sensing circuitry, whether the metric of the patient's autonomic function indicates a risk of overregulation, by identifying a first marker of autonomic function of the patient, obtaining an activity signal from the patient indicating a level of intentional activity by the patient, and determining an effect on the first marker caused by intentional activity of the patient. Additionally or alternatively, the control circuitry is configured to obtain the activity signal from the patient by using an accelerometer or by monitoring a component of the electrospinogram.

Additionally or alternatively, the control circuitry is configured to reduce the risk of overregulation by reducing one or more of an intensity, a repetition rate, a pulse width, or an amplitude of the pulses delivered to the patient, and/or the control circuitry is configured to reduce the risk of overregulation by ceasing to issue pulses to the patient in accordance with the first treatment program. Additionally or alternatively, the control circuitry is further configured to issue pulses to the patient in accordance with a second treatment program after ceasing to issue pulses to the patient in accordance with the first treatment program.

Additionally or alternatively, the system may also include a patient remote control external of the patient having a user interface and communication circuitry for communicating with the pulse generator, wherein the control circuitry is configured to reduce the risk of overregulation by communicating to the remote control to present a query to the patient regarding stimulation the patient is receiving.

Additionally or alternatively, the control circuitry is configured to reduce the risk of overregulation by issuing a communication with an alert that the patient is at risk of overregulation.

Additionally or alternatively, the sensing circuitry is contained in the pulse generator housing and communicates directly with the control circuitry, and/or the sensing circuitry is contained in a separate wearable or implantable medical device, and the separate wearable or implantable medical device and the pulse generator each include communication circuitry configured such that the separate wearable or implantable medical device can communicate data from the sensing circuitry to the control circuitry.

Another illustrative and non-limiting example takes the form of a method of treating a patient having an implantable medical device system including a lead adapted for placement in the spinal column of a patient and a housing containing each of a sensing circuitry configured to sense a metric of the patient's autonomic function, and a stimulation circuitry configured to issue pulses via the lead; the method comprising: issuing pulses to the patient in accordance with a first treatment program; determining, from the sensing circuitry, whether the metric of the patient's autonomic function indicates a risk of overregulation; and in response to a determination that the metric of the patient's autonomic function indicates a risk of overregulation, reducing the risk of overregulation.

Additionally or alternatively, the step of determining, from the sensing circuitry, whether the metric of the patient's autonomic function indicates a risk of overregulation, is performed by tracking a trend the metric.

Additionally or alternatively, the step of determining, from the sensing circuitry, whether the metric of the patient's autonomic function indicates a risk of overregulation, is performed by: detecting, using the sensing circuitry, heart beats of the patient; determining, from the detected heart beats, a cardiac rate of the patient; and comparing the cardiac rate or a trend of the cardiac rate to a threshold.

Additionally or alternatively, the step of determining, from the sensing circuitry, whether the metric of the patient's autonomic function indicates a risk of overregulation, is performed by: detecting, using the sensing circuitry, heart beats of the patient; determining, from the detected heart beats, a heart rate variability of the patient; and comparing the heart rate variability or a trend of the heart rate variability to a threshold.

Additionally or alternatively, the step of determining, from the sensing circuitry, whether the metric of the patient's autonomic function indicates a risk of overregulation, is performed by: detecting, using the sensing circuitry, a respiration rate of the patient; comparing the respiration rate, or a trend of the respiration rate, to a threshold.

Additionally or alternatively, the step of determining, from the sensing circuitry, whether the metric of the patient's autonomic function indicates a risk of overregulation, is performed by: detecting, using the sensing circuitry, heart beats of the patient; analyzing the heart beats of the patient to yield a first estimate of the patient's autonomic state; obtaining data from a second device to yield a second estimate of the patient's autonomic state, wherein the second device provides an indication of blood pressure. The determining step may include as well observing outputs of an accelerometer or patient position sensor to determine if the patient has moved from one position to another, such as by standing up from a sitting position, which can affect the monitored metric.

Additionally or alternatively, the step of determining, from the sensing circuitry, whether the metric of the patient's autonomic function indicates a risk of overregulation, is performed by: detecting, using the sensing circuitry, heart beats of the patient; analyzing the heart beats of the patient to yield a first estimate of the patient's autonomic state; obtaining data from a second device to yield a second estimate of the patient's autonomic state, wherein the second device provides an indication of galvanic skin resistance of the patient.

Additionally or alternatively, the step of determining, from the sensing circuitry, whether the metric of the patient's autonomic function indicates a risk of overregulation, is performed by: determining a first autonomic function marker and a second autonomic function marker each selected from heart rate, heart rate variability, blood pressure, respiration rate, galvanic skin response, bladder, bowel or sexual function, posture and myopotentials; and using the first autonomic function marker and the second autonomic function marker to estimate the metric.

Additionally or alternatively, the step of determining, from the sensing circuitry, whether the metric of the patient's autonomic function indicates a risk of overregulation, is performed by: identifying a first marker of autonomic function of the patient, obtaining an activity signal from the patient indicating a level of intentional activity by the patient, and determining an effect on the first marker caused by intentional activity of the patient. Additionally or alternatively, the step of obtaining an activity signal is performed by using an accelerometer in the sensing circuitry. Additionally or alternatively, the step of obtaining an activity signal is performed by monitoring a component of the electrospinogram.

Additionally or alternatively, the step of reducing the risk of overregulation is performed by reducing one or more of an intensity, a repetition rate, a pulse width, or an amplitude of the pulses delivered to the patient. Additionally or alternatively, the step of reducing the risk of overregulation is performed by ceasing to issue pulses to the patient in accordance with the first treatment program. Additionally or alternatively, the method may include, after ceasing to issue pulses to the patient in accordance with the first treatment program, issuing pulses to the patient in accordance with a second treatment program.

Additionally or alternatively, the implantable medical device system further includes a patient remote control external of the patient having a user interface and communication circuitry for communicating with the pulse generator, wherein the step of reducing the risk of overregulation is performed by communicating to the remote control to present a query to the patient regarding stimulation the patient is receiving.

Additionally or alternatively, the step of reducing the risk of overregulation is performed by issuing a communication with an alert that the patient is at risk of overregulation.

Another illustrative and non-limiting example takes the form of a method of treating a patient using a spinal cord stimulator, comprising: generating a mapping of autonomic status of the patient using a first metric and a second metric, the mapping including a first region for a stressed status, and a second region for a relaxed status; initiating therapy using a first therapy program; confirming, by tracking the first metric and the second metric, that the patient's autonomic status has shifted from the first region to the second region in response to initiating therapy using the first therapy program; monitoring the patient's autonomic status in the second region as therapy continues using the first therapy program; determining that the patient's autonomic status has left the second region; in response to the determining that the patient's autonomic status has left the second region, determining that the patient is overregulated.

Additionally or alternatively, the method includes, in response to determining that the patient is overregulated, reducing one or more of an intensity, a repetition rate, a pulse width, or an amplitude of pulses delivered to the patient in the first therapy program.

Additionally or alternatively, the method includes, in response to determining that the patient is overregulated, terminating therapy using the first therapy program, and initiating therapy using a second therapy program.

Additionally or alternatively, the first metric is heart rate, and the second metric is heart rate variability. Additionally or alternatively, the first metric and the second metric are selected from heart rate, heart rate variability, blood pressure, respiration rate, galvanic skin response, bladder, bowel or sexual function, posture and myopotentials.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows components of an illustrative neuromodulation system;

FIG. 2 shows an illustrative pulse generator and lead system;

FIG. 3 shows a spinal cord stimulation system as implanted in a patient;

FIG. 4 illustrates how cardiac signals may be monitored with a spinal cord stimulation system;

FIG. 5 shows a response of a patient to neuromodulation in terms of cardiac rate and heart rate variability; and

FIG. 6 is a block flow diagram for several illustrative examples.

### DETAILED DESCRIPTION

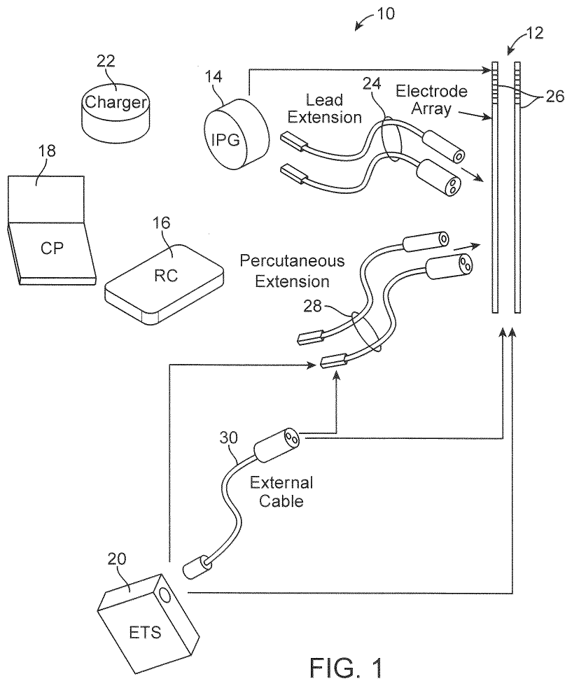

FIG. 1 shows a system for providing neurological therapy, which may be used, for example, as spinal cord stimulation (SCS), deep brain stimulation (DBS), peripheral nerve stimulation (PNS), or functional electrical stimulation (FES). The system 10 includes leads 12 configured for coupling to an implantable pulse generator (IPG) 14. The IPG may communicate with one or more of a patient remote control (RC) 16, a clinician programmer (CP) 18, and/or a charger 22. An external testing system (ETS) 20 may also be provided for testing therapy parameters prior to implantation of the IPG, using percutaneous extensions 28 and, as needed, an external cable 30 to couple to the leads 12. The use of an ETS may be referred to as a trial period. If needed, lead extensions 24 may be used to couple the IPG to the leads 12.

As shown in FIG. 1, the leads 12 may include arrays of electrode contacts on linear leads 26; in other examples, paddle leads may be used. One, two or even four leads 12 may be provided, with up to 32 contacts on the leads 12, plus an additional contact in the form of the housing of IPG 14, available in modern systems. More or fewer contacts on more or fewer leads may be provided depending the particular system.

The IPG 14 can couple directly to the leads 12 or may be coupled via the lead extensions 24, depending on the positioning of each element as implanted. The IPG 14 may include a rechargeable battery and charging coil to allow recharging when placed in proximity to the charger 22. Alternatively, the IPG may use a non-rechargeable battery and omit a charging coil and charger 22 from the system. In some examples, the IPG 14 may be externally powered and omits a battery entirely.

The CP 18 can be used by a physician to manipulate the outputs of the IPG 14 and/or ETS 20. For example, the CP 18 can be used by the physician to define a therapy regimen or program for application to the patient. The CP 18 may be a custom device and/or may take the form of a laptop or tablet computer, for example and without limitation. Multiple programs may be facilitated and stored by the IPG 14 or ETS 20; in some examples, the RC 16 may store the programs to be used. Communication amongst the IPG 14, RC 16, CP 18, ETS 20 and Charger 22 may use any suitable protocol such as wireless RF telemetry, Medradio, inductive communication, Bluetooth, etc.

The RC 16 may be used by a patient to enable or disable therapy programs, to select between available programs, and/or to modify the programs that are available for use. For example, in some embodiments a patient may use the RC 16 to activate a stored program and then manipulate therapy by increasing or decreasing therapy strength and/or changing therapy location, within limits set by the physician. The RC 16 may be a custom device, or may be, for example, a smartphone or table having an application thereon for use with the medical device system 10.

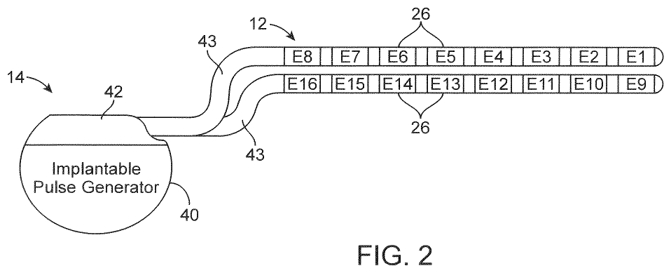

FIG. 2 shows an implantable stimulator and leads. As shown in the closer detail here, the IPG 14 may include a canister 40 and header 42. The canister 40 is conductive in most examples, using biocompatible materials such as titanium and/or stainless steel, for example, to allow use as an electrode when implanted. The header 42 allows removable connection to the lead 12, which in this example may have a bifurcation or yoke allowing two segments 43 to extend therefrom, to two arrays 26 at the distal end of the lead 12. A common structure for securing the leads 12 is the use of a setscrew in the header 42. The electrode arrays 26 can be numbered as shown to facilitate ease of understanding when programming, with, for example, one array marked electrodes E1 to E8 on one of the leads 43, with E1 being distalmost. Other conventions may be used.

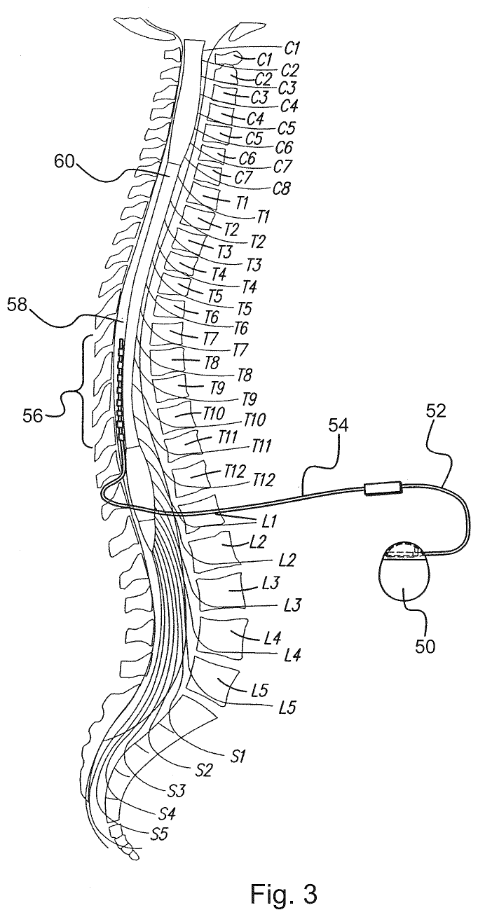

FIG. 3 shows an illustrative spinal cord stimulation system as implanted. In this example, an IPG 50 may be placed near the buttocks or in the abdomen of the patient, with or without a lead extension 52 for coupling to the lead(s) 54 that enter the spinal column. Region 56, at about the level of the lower thoracic or upper lumbar vertebrae may serve as an entry point to the spinal column, where the distal end of the lead 54 with an electrode array may be placed close to the spinal cord 58. Other locations for the IPG 50 and/or lead 54 may be used.

The standard approach to therapy in systems similar to those shown in FIGS. 1-3 has been that the IPG 14, 50, or ETS 20, may offer current controlled or voltage-controlled therapy comprising either biphasic square waves or monophasic square waves having passive recovery. In general, the amount of current out of an electrode should zero out over time to avoid encouraging corrosion at the electrode-tissue interface. For this reason, biphasic pulses, or monophasic pulses with a passive recovery period are typically used.

The leads 54, as shown in FIG. 3, are often placed in the thoracic region of the spinal column. Afferent signals, such as pain signals, transmit toward the brain along the spinal column. Some such signals do not enter the spinal column at or caudal to the thoracic lead position usually used. As a result, SCS delivered in the thoracic spine cannot affect pain signals that reach the spinal column at such higher positions.

Much of the therapy delivered in SCS is provided with no feedback or only indirect feedback. For example, during some testing and configuration procedures, a patient may consciously respond to questions regarding therapy benefits or side effects, interposing both the delay time needed for a patient to perceive stimulus effects, as well as adding uncertainty due to subjective factors to the feedback. Some side effects of SCS therapy may not be immediately apparent to the user and or physician, and may build over time. For example, an SCS therapy initiated at a given time may provide therapy, such as pain relief, for a period of time, however, after active operation for an extended period (minutes to hours), the therapy may no longer be comfortable, or the patient may experience changes in sympathetic or parasympathetic tone indicating that the patient is becoming overstimulated or, as used herein, overregulated. An SCS system that can identify indications of overregulation is desired. The following examples show several systems configured for sensing autonomic nervous system indicators and using such indicators to titrate SCS therapy.

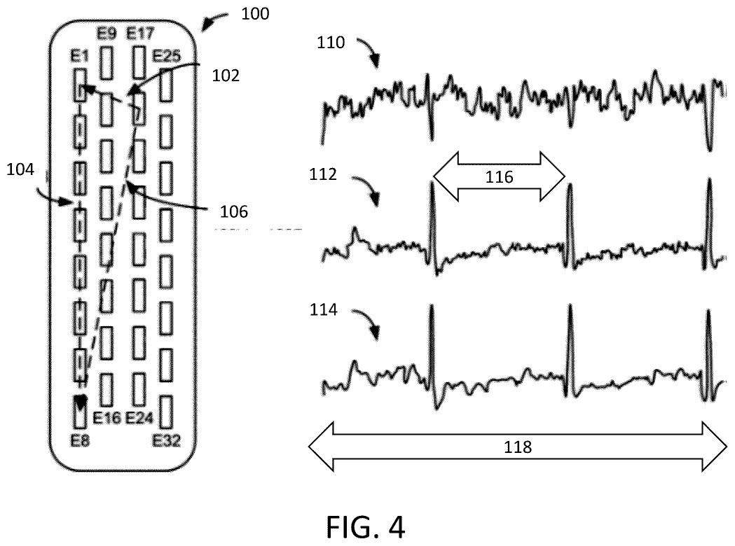

FIG. 4 illustrates how cardiac signals may be monitored with a spinal cord stimulation system. A distal end of a paddle lead is those at 100, having four columns of electrode contacts numbered E1 to E32. Only the end electrodes are marked, but the numbering scheme proceeds from E1 to E8 along the leftmost column, from E9 to E16 at the center-left column, from 17 to E24 in the center-right column, and from E25 to E32 on the right-most column.

Sensing data may be captured between pairs of electrode contacts to generate sensing “vectors,” with a first sensing vector between electrodes E1 and E18 (shown at 102), a second sensing vector between electrodes E1 and E8 (shown at 104), and a third sensing vector between electrodes E8 and E18 (shown at 104). Each sensing vector provides a different electrical “view” of the cardiac signal. Vector between more spaced electrodes (vectors 104 and 106) may receive a larger amount of noise, but higher overall signal, than vectors between more closely spaced electrodes. The sense data may be obtained by filtering (such as DC blocking) raw signal received between the electrodes, amplifying, digitizing by analog-to-digital conversion, and then digitally filtering the signals, as are known in the art. Detailed discussions of some methods for obtaining a cardiac signal using spinal-cord-positioned electrodes appear in U.S. Pat. No. 10,974,042, the disclosure of which is incorporated herein by reference.

After various filtering exercises, the resultant cardiac signals may appear as shown on the right side of FIG. 4. Three different signals are shown, each corresponding to one of the sensing vectors. Signal 110 is relatively disorganized, with some characteristic spikes that may indicate cardiac activity. Signal 112 is more organized, including distinct spikes which may correspond to the R-waves of the electrocardiograph (ECG), which, according to accepted convention, indicate electrical signals generated with ventricular contraction. Likewise, signal 114 provides a relatively organized view, again with distinct spikes that may correspond to the R-waves of the cardiac signal.

In some examples, the cardiac R-wave peaks are analyzed to determine the R-R interval 116, the inverse of which is the cardiac rate. For example, if the R-R interval 116 is 800 milliseconds long, then the patient has a cardiac rate of 75 beats per minute. R-R intervals may be monitored and stored over time to determine heart rate variability (HRV), which is an indicator of neural function. A relatively lower pulse rate with higher HRV may indicate increased parasympathetic function, indicating a greater degree of relaxation, while a higher pulse and lower HRV may indicate increased sympathetic function, indicating the body is on higher alert or tenser. Increased sympathetic function may be an indication of overregulation, or ineffective under-regulation. Increased parasympathetic function may be an indication of appropriate regulation or modulation.

FIG. 4 provides illustrations of how an SCS system may monitor cardiac data. In some examples, rather than the SCS system monitoring the heart rate and/or HRV, a second device such as a wearable Holter device, other wearable cardiac monitor, or an implantable device (such as a cardiac monitor, pacemaker or defibrillator) may be used. Systems directed to heart monitoring may be able to provide high accuracy indications of factors like rate and HRV.

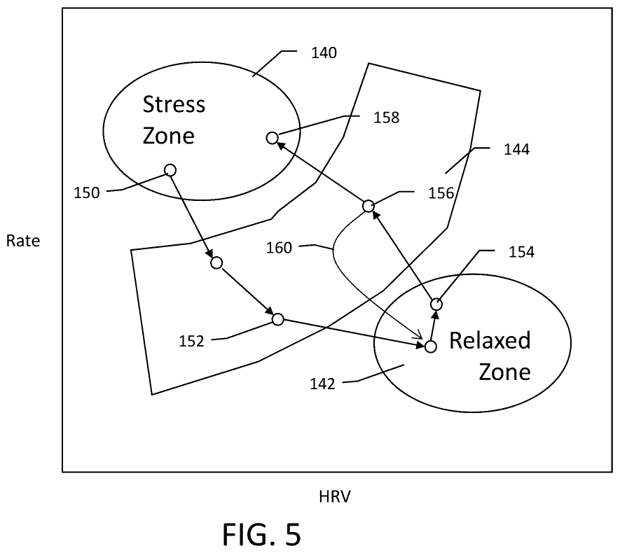

FIG. 5 shows a response of a patient to neuromodulation in terms of cardiac rate and heart rate variability. Cardiac rate and heart rate variability (HRV) form the vertical and horizontal axes, respectively, as indicated. A stress zone 140, in this instance (though not limited as such) generally linked to greater sympathetic function is indicated, with relatively higher heart rates and lower HRV. A relaxed zone 142, in this instance generally (though not limited as such) linked to greater parasympathetic function is indicated, with relatively lower heart rates and higher HRV. A border 144 between stress zone 140 and relaxed zone 142 is roughly defined as indicated at 144. The boundaries for each of zones 140 and 142 may be determined for a particular patient in a controlled setting, if desired, or may be obtained from any suitable reference (such as population-based data). For example, the patient may be provided with effective, pain relieving therapy in a controlled setting while stationary to identify data points within the relaxed zone 142, after a baseline setting in which the patient is not treated, with the baseline used to establish stress zone 140. Any suitable method of identifying the outer boundaries of zones 140 and 142 may be used, such as by setting a +/−percentage of each of rate and HRV for each range, or by use of standard deviations from observed points in each range, for example. The border 144 therebetween can simply be the space not otherwise within one of the zones 140, 142.

A first patient response to therapy is indicated as a path from the stress zone 140 starting at point 150. When therapy is initiated, the patient in this path experiences relief from symptoms, such as relief from pain, and the body's response as determined from heart rate and HRV proceeds to point 152 in the border 144, then to the relaxed zone 142, residing for a period of time in the relaxed zone 142, as indicated at point 154. Other terminology can be used; relaxed zone 142 may be understood as desirable stimulation, optimal stimulation, or the like. It is common that a patient begins to feel overregulated after a period of time, and the body may indicate this overregulation before the patient consciously notices it. In the example, the body's response changes over time, and heart rate increases and HRV decreases, as indicated by the path continuing to the border 144 with point 156, and then returning to the stress zone 140 at point 158. At this point 158, the patient may feel uncomfortable, even if the delivered SCS therapy is not necessarily identified by the patient as the source of the shift.

Some examples of the present invention may identify one or more of the initial portion of the path toward the relaxed zone 142, with reduced heart rate and increased HRV. Some examples may also identify the time spend in the relaxed zone 142, and may also identify when the patient's detected body conditions shift to the border 144 and even to the stress zone 140. In an example, when the patient's detected rate and HRV indicate a shift from the relaxed zone 142 to the border 144, therapy parameters are changed, such as by reducing amplitude or pulse width, with the aim to return the patient to the relaxed zone 142, as indicated by line 160. In another example, therapy may cease, such as by terminating therapy entirely, or by transitioning from a first therapy program to a second therapy program. In still another example, therapy may be modified when it is determined that the patient has exited zone 142 and entered border 144, as a first response, and if the patient continues toward the stress zone 140, therapy may be turned off once zone 140 is entered. Other examples are further discussed below.

The axes may be changed for other sensed parameters. In general, the concept of defining “stress” (zone 140), “borderline” (region 144), and/or “Relaxed” (zone 142) zones can be applied to other metrics, such as blood oxygenation, glucose levels, and breathing or perspiration sensing (QD). Heart rate and HRV are used in FIG. 5 as an illustration of the workflow. Any of these other markers, alone or taken in any desired combination of two or more markers, may be used in similar or analogous fashion as a metric of the patient's autonomic function or state. The position of the metric as plotted on such axes is an indication of the patient's autonomic function. A position, change in position, or trend or path of positions of the metric is used in some examples to indicate a risk of overregulation. For example, a risk of overregulation may be identified at least when the position of the metric is outside of the relaxed zone 142. In other examples a risk of overregulation can be identified if the metric is plotted inside the stress zone 140. In further examples, a direction of change of the metric toward the stress zone indicates a risk of overregulation. In some examples, a plotted path or trend of the metric that enters the relaxed zone and later exits while stimulation is still occurring indicates a risk of overregulation. Risks of under-regulation may also be identified, such as when the plotted metric is in the stressed zone 140, outside of the relaxed zone 142, trending toward the stressed zone 140, and/or, after turning stimulation on, the patient's metric never leaves the stressed zone 140. Optimized stimulation may be identified as within range when the autonomic measures sit between high and low boundaries each associated with over or under regulation. Optimized stimulation may be identified if the patient's metric takes a path that leaves the stressed zone 140 when stimulation is turned on, enters the relaxed zone 142 and remains in the relaxed zone over time. In another example, optimized stimulation is identified if the metric path exits the stressed zone 140 after stimulation turns on, but does not return, though the path may exit and then reenter the relaxed zone 142 over time, for example.

Metrics may be plotted in a single dimension (as on a line), in two dimensions as illustrated in FIG. 5, or in more than two dimensions, as desired. Distance to the stress or relaxed zone can be determined using vector math, with normalization applied to different metrics in accordance with any suitable determinations. “Distance” may be used to aid in understanding path and direction of a trend; for example, if multidimensional metrics are used with two or more components, one component may move toward a relaxed state while another moves toward the stress zone, and standard vector math can be used to estimate the direction of a trend, as desired and suitable to the particular application, patient, and/or physician preferences.

For example, blood oxygenation may be monitored by an SCS system itself or, more likely, by a second system (implanted or wearable). When therapy initiates to treat, for example, pain via SCS, blood oxygenation is likely to increase, as the patient experiences relief and begins to breathe with a normal cadence and depth. If therapy is insufficient, a response of increased blood oxygenation may not occur, and so therapy intensity may be increased by the system. On the other hand, if blood oxygenation increases and, after some period of time, blood oxygenation starts to decrease again, with or without another marker, it may be concluded that the patient has become overregulated, and stimulation intensity (such as amplitude) may be decreased, stopped, or the system may switch to a different programmed therapy. In this way, blood oxygenation, alone or in combination with another marker, may be a metric of the patient's autonomic function.

For another example, blood glucose may be monitored by an SCS system itself or, more likely, by a second system (implanted or wearable). When therapy initiates to treat, for example, pain via SCS, blood glucose is likely to be reduced, as the patient experiences relief and the sympathetic nervous system stops generating hormones that cause blood glucose to increase as a stress response. If therapy is insufficient, a response of decreased blood glucose may not occur, and so therapy intensity may be increased by the system. On the other hand, if blood glucose decreases and, after some period of time, blood glucose starts to increase again, with or without another marker, it may be concluded that the patient has become overregulated, and stimulation intensity (such as amplitude) may be decreased, stopped, or the system may switch to a different programmed therapy. In this way, blood glucose, alone or in combination with another marker, may be a metric of the patient's autonomic function.

For another example, blood pressure may be monitored by an SCS system itself or, more likely, by a second system (implanted or wearable). When therapy initiates to treat, for example, pain via SCS, blood pressure is likely to be reduced, as the patient experiences relief and the sympathetic nervous system stops generating hormones that cause blood pressure to increase as a stress response. In some examples, a particular portion of the blood pressure, signal, such as systolic pressure or diastolic pressure, or both. If therapy is insufficient, a response of decreased blood pressure may not occur, and so therapy intensity may be increased by the system. On the other hand, if blood pressure decreases and, after some period of time, blood pressure starts to increase again, with or without another marker, it may be concluded that the patient has become overregulated, and stimulation intensity (such as amplitude) may be decreased, stopped, or the system may switch to a different programmed therapy. The analysis may include as well observing outputs of an accelerometer or patient position sensor to determine if the patient has moved from one position to another, such as by standing up from a sitting position, which can affect the monitored metric. In this way, blood pressure, alone or in combination with another marker, may be a metric of the patient's autonomic function.

In another example, breathing may be monitored by an SCS system itself, or by a second system. For example, breathing can be monitored by the SCS system using one or more of thoracic impedance (including oscillations thereof), changes in sensed signals (such as components of the electrospinogram, including stimulation artifact, evoked compound action potentials, or background potentials) along the spinal column (which change due to minute movements caused by inspiration and expiration), by monitoring for the sounds associated with breathing, or by monitoring for electrical signals emanating as the diaphragm contracts, for example. Breathing may instead be monitored by a second system and breathing characteristics (rate, depth, for example) may be communicated to the SCS system. When the patient is in a stressed state, breathing tends to be shallower and more rapid. When therapy initiates to treat, for example, pain via SCS, breathing rate can be expected to reduce, and/or the depth of inspiration in particular can be expected to increase if the patient experiences relief from pain. Depth of inspiration may be observed, for example, from extremum conditions (thoracic impedance for example) and or the duration of signals from the diaphragm, noise, or other measures, while frequency can be observed based on time between signal cycles. If therapy is insufficient, a response of decreased breathing rate and/or increased depth of breathing may not occur, and so therapy intensity may be increased by the system. On the other hand, if breathing rate decreases and/or breathing depth increases and, after some period of time, breathing rate begins to increase and/or breathing becomes shallower, again, with or without another marker, it may be concluded that the patient has become overregulated, and stimulation intensity (such as amplitude) may be decreased, stopped, or the system may switch to a different programmed therapy. In this way, breathing, alone or in combination with another marker, may be a metric of the patient's autonomic function.

In another example, the output of a wearable sweat sensor, or galvanic skin response can be monitored by a wearable system, thus checking for perspiration, with results sent to the SCS system. When therapy initiates to treat, for example, pain via SCS, perspiration is likely to be reduced, as the patient experiences relief and the sympathetic nervous system stops generating hormones that cause perspiration to increase as a stress response. If therapy is insufficient, a response of decreased perspiration may not occur, and so therapy intensity may be increased by the system. On the other hand, if perspiration decreases and, after some period of time, perspiration starts to increase again, with or without another marker, it may be concluded that the patient has become overregulated, and stimulation intensity (such as amplitude) may be decreased, stopped, or the system may switch to a different programmed therapy. In this way, perspiration, alone or in combination with another marker, may be a metric of the patient's autonomic function.

Posture may also be monitored, as by the monitoring of stimulation artifacts in SCS, components of the electrospinogram, including stimulation artifact, evoked compound action potentials, or background potentials, and/or measurements obtained from an accelerometer in the SCS system itself or in another implantable or wearable device. This may be used, for example, as a secondary check to determine if the patient is at rest while an elevated heart rate occurs, indicating a stress-related cause for the elevated rate. Posture can also be relevant to blood pressure, for example, in cases of orthostatic hypotension and/or syncope, each of which may indicate an imbalance between sympathetic and parasympathetic function, addressable by stimulation. In this way, posture, alone or in combination with another marker, may be a metric of the patient's autonomic function.

Respiration rate may also be used in conjunction with HRV to monitor respiratory sinus arrhythmia (RSA). RSA indicates heart rate variability in synchrony with respiration, by which the R-R interval is shortened during inspiration and prolonged during expiration. RSA may be “measured” as the difference between the average R-R interval during inspiration and the average R-R interval during expiration, and can be scaled to a beats-per-minute measure. An RSA of 15 bpm or above is generally considered “normal,” though other levels may be used. In some examples, a patient's RSA can be determined when the patient is at rest and receiving pain-relieving therapy in clinic, for example, or other controlled environment, to determine a reference to use for characterizing the patient's RSA as normal or low. Normal RSA indicates the patient is in a relaxed or un-stressed state, and low RSA indicates stress. Deviations from the patient's typical range of RSA, particularly a decrease in RSA with extended SCS duration, are used in some examples to identify overregulation. In this way, RSA, alone or in combination with another marker, may be a metric of the patient's autonomic function.

Patient temperature may be monitored, if desired, using an implantable or wearable device to observe changes in patient temperature. Increase or decrease of the sensed temperature may likewise be subject to the trends and changes for other parameters.

Skeletal muscle activity may also be monitored via, for example, sensed by electromyography (EMG). For example, an SCS system may monitor skeletal muscle noise in the same way that a cardiac signal is obtained, generally, except that filtering to obtain the target signal would be altered. The cardiac signal is typically in the range of about 3 to 60 Hz (and ventricular signals of typical interest tend to be in the range of 3-40 Hz, as discussed in US PG Pub. US20210113135A1), though wider ranges can be used. Skeletal muscle signals may appear at relatively higher frequencies, over 60 Hz up to close to 1 kHz, generally. Monitoring, for example, the strength of root-mean-squared (RMS) signal band-passed to a range of 100 Hz to 1 kHz, with line noise (50/60 Hz, depending on geography) strongly filtered out, can provide a general understanding of how much skeletal muscle activity is occurring, an indication of tension particularly when the patient is not moving. When SCS is initiated, if pain relief is experienced, is may be expected that the skeletal muscle signal would reduce in amplitude as tension is released. If therapy is insufficient, a response of decreased skeletal muscle signals may not occur, and so therapy intensity may be increased by the system. On the other hand, if skeletal muscle signals decrease and, after some period of time, start to increase again, with or without another marker, it may be concluded that the patient has become overregulated, and stimulation intensity (such as amplitude) may be decreased, stopped, or the system may switch to a different programmed therapy. In this way, skeletal muscle activity, alone or in combination with another marker, may be a metric of the patient's autonomic function.

In some cases, the system monitors a sensed signal correlated to patient stress to observe a reduction in the signal in response to therapy, and later increase indicating, via the metric of autonomic function, possible onset of overregulation. On the other hand, if the sensed signal instead increases (such as increases skeletal muscle activity) acutely in response to stimulation initiation, the patient may be experiencing an acute overregulation event. The system may respond to an identified acute overregulation event by interrupting the stimulation signal, or reducing at least one of amplitude and/or intensity.

Some examples that use a second device to obtain data indicating patient stress levels and/or other metrics of autonomic function may use any wearable device the patient has or uses, particularly if available on a regular basis. For example, a patient may have a wearable brain function monitor that can be used as a second device.

In any of these examples, in addition to or in place of increasing or decreasing intensity by increasing or decreasing amplitude, any of pulse width, duty cycle, and/or frequency/repetition rate may be increased or decreased as a way to change intensity. Further, in any of these examples, additional criteria may be included to rule out other, non-stimulation-related, possible causes of a stress response. For example, if the system includes an activity monitor (internal accelerometer, for example) or is operably linked to a second device, such as a wearable activity monitor (fitness watch, for example), the system may first rule out an increase in activity, initiation of exercise for example, before concluding that the patient is potentially overregulated.

The plot as shown in FIG. 5 may be generated during in-clinic and/or trial stimulation (external stimulator or ETS, as shown in FIG. 1). The patient may be instructed as to the meaning of the zones, and this may be a useful way to inform the patient about how their system works. Such a plot may be displayed, for example, on the patient RC and/or on a clinician programmer or CP as a guide for user modifications (via the RC) of therapy amplitude, and/or for guiding clinical programming. The plot may instead be displayed on a separate or associated application operating on the CP and/or RC, or entirely separately on a different device, as desired.

For example, a patient may be allowed to adjust therapy amplitude using the RC. A plot as shown in FIG. 5 may be shown to the patient by the RC at a given point in time when the patient is trying to adjust therapy amplitude, as a way of either suggesting a change to the patient, or to dissuade the patient from making a change that could be undesired (such as increasing amplitude of stimulation when it is likely to drive the patient toward a state of stress such as the stress zone 140.

In some examples, the system may track the path in order to determine what to do in response to being outside of the relaxed zone 142. For example, if stimulation starts and the patient is observed to be in the stress zone 140 and sensed activity indicates the patient is in the border zone 144, but has not reached the stress zone 140, this may be because the patient is under-regulated, so amplitude can be increased. On the other hand, if the patient is observed to have been in the relaxed zone 142 for a period of time, but the sensed data indicates that the patient is no longer in zone 142 and has entered the border 144, this may be because the patient has become overregulated, and so amplitude can be decreased.

As noted, there may be multiple markers available for use in a system's metric of autonomic function. A user (clinician or patient) may be presented with a list of potential markers that can be used to monitor stress response. Such selections may be informed by patient disease state/type and/or any secondary devices that are present; for example, if a glucose monitor is present for the patient and/or the patient has diabetes, blood glucose may be a likely marker to use. If the patient has a cardiac monitor, pacemaker or defibrillator, then heart rate and/or HRV may be used.

Different weights may be used to emphasize or de-emphasize markers based on user input. Combinations of factors may increase confidence in system determinations, so that a closed loop control is effectuated using markers that are distinct from those directly related to the therapy itself, such as the monitoring of components of the electrospinogram, including stimulation artifact, evoked compound action potentials, or background potentials, responsive to therapy delivery. In some examples, a response to one of the above markers may include presenting a stress-related query to a patient via the patient RC or by an application operated on a patient mobile device (smartphone for example), allowing the system to confirm with the patient that the patient is feeling a greater level of stress than desired. Such patient feedback may include queries regarding the sensation of any pain spikes, for example, or anxiety, depression, emotional, psychosocial or other state of the patient. Other bodily function queries may be presented including those related to any of bladder, bowel or sexual function. Any such feedback may be compared to patient-specific baseline data, population-based data, or general guidelines.

Several of the preceding examples indicate a change in therapy intensity, which can be implemented in several ways. In some examples, a user, such as the patient, a caregiver, or a clinician, may be informed of the observations regarding sympathetic function that a system makes, as by communication via the RC or CP, or by an associated application, for example. Decisions regarding whether to make a change in intensity, and how, are then left to the user. In other examples, when the user is provided such information, one or more therapy change recommendations changes may also be presented to the user for approval. In still other examples, the system may simply change the therapy itself.

In some further examples, if the therapy then ongoing is changed, a stored set of parameters for a therapy program may also be revised, or requested (to a user) to be revised, for a future activation of the therapy program. That is, the current, in-use, program may be changed as a response to sensed activity, and the program that is in use may also be modified so that a later use of the same program will be less likely to cause overregulation (or under-regulation, depending on the type of revision). Such usage changes may include, for example, modifying any of amplitude, pulse width, duty cycle, repetition rate, maximum duration that a therapy program may be active, or any other desired parameter. Usage changes may also be performed to determine whether patient adjustments to therapy should be limited. For example, it may be that a program as stored in the system is adjusted by a user increasing amplitude before overregulation occurs; the user option to increase amplitude may be taken away or capped if overregulation occurs.

The examples just described presume a relatively short time period between stimulation, and stimulus changes, and patient physiological response. Some changes, however, may occur only hours or even days later. Monitoring for much later changes may be performed and recorded data stored for later review by a clinician, if desired. Furthermore, the system may be adapted to analyze such longer-term data, such as over hours, days or even weeks, to identify changes and relate such changes back to prior events. For example, if a patient increases therapy amplitude, where the therapy has been used for weeks or months at a relatively lower amplitude, and after a few hours or days the patient becomes stimulated, the change in amplitude may be linked by the device searching for any prior changes. Further, the patient may respond to various requests from the RC for information, which the device may also use as an indication of a change. For example, if a patient's medications change, such a change may be observed by the device receiving patient inputs in response to prompts from the RC. If a later overregulation event occurs, days, or weeks after the medication change, the device can flag the patient-indicated change as a potential cause or source. The system may, in response to such determinations, present or revise therapy recommendations to prevent reoccurrence in future application.

Some examples may include the use of sub-perception or paresthesia-free therapy. For sub-perception therapy, for example, a fitting approach may include mapping paresthesia coverage over a pain region, and then reducing amplitude until the paresthesia is no longer observed. For such patients, the ability to monitor for therapy efficacy may be somewhat masked by the absence of paresthesia. The use of a monitoring approach for autonomic nervous system markers just described may be applicable to such patients, and additional steps may be included. For example, testing of the paresthesia threshold may be performed by raising therapy amplitude temporarily until the patient experiences paresthesia, such as in an RC-modulated test regimen. Doing so may confirm the appropriate stimulation amplitude, as well as confirming that stimulation is spatially appropriate if the patient senses the paresthesia as being located at/over source of pain. Rather than relying on the patient to indicate paresthesia, components of the electrospinogram, including stimulation artifact, evoked compound action potentials, or background potentials may be monitored, allowing self-test by the implanted device without patient involvement. When the monitored autonomic system markers of the patient indicate the patient is in the relaxed zone, such periodic testing may be skipped or omitted, if desired. If the patient is observed, using autonomic system markers, to have left the relaxed zone, then the testing of the paresthesia may be triggered or, if performed periodically, made more frequent.

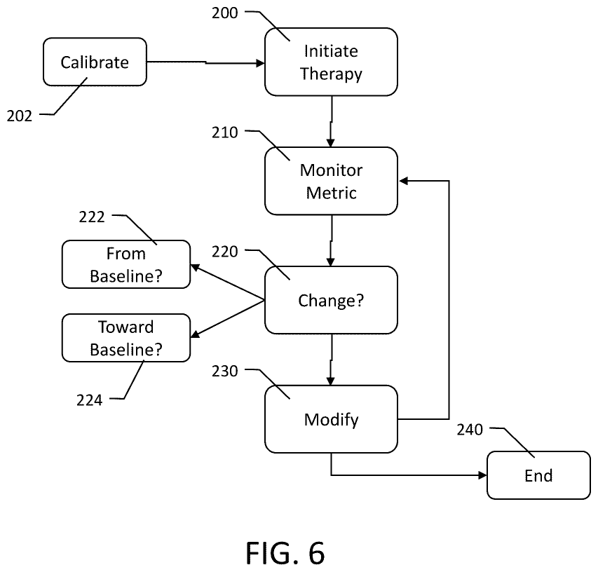

FIG. 6 is a block diagram for an illustrative method. Therapy is initiated at 200, which may include initiating a therapy program for SCS. Calibration 202 may be a predicate, if desired. The system then monitors one or more metrics of autonomic function, as indicated at 210. The system determines whether the metric(s) of the patient's autonomic function has changed, as indicated at 220. The system then determines whether to modify therapy, as indicated at 230. Block 230 may revert to block 210, as shown, for continued monitoring. If a modification is made at 230, this may include terminating therapy, as indicated at 240.

Going back to each block for further detail, the monitoring of the metric of the patient's autonomic function 210 may include the use of any one or several of the preceding examples. Thus block 210 may include monitoring any of heart rate, heart rate variability, respiratory sinus arrhythmia, skeletal muscle signals, posture or movement, respiration, perspiration, blood oxygenation, blood pressure, blood glucose, blood oxygenation, and/or other markers described above singly or in combinations and/or weighted combinations.

The monitoring in block 210 may be aided by calibration 202. Calibration 202 may be performed in a controlled environment, if desired, though this need not be the case. Calibration may include observing what happens to selected metrics of autonomic function when therapy is turned on while the patient is being directly monitored by a physician or other clinician, or while interacting with a patient RC. Calibration 202 may be used to set boundaries as illustrated in FIG. 5, for example.

Determining whether a metric of autonomic function has changed at 220 may have several aspects. When therapy first starts, the change monitored at 220 may be to ensure a departure from the pre-therapy baseline, as indicated at 222. If no such change, then therapy can be modified at 230 to increase intensity, for example. After the change from baseline 222 is observed, then the change to monitor is whether the patient is starting to move back toward the baseline, as indicated at 224. Change monitoring may also consider whether the desired autonomic state has been achieved at all. For example, referring to FIG. 5, if the patient never reaches zone 142, a modification to therapy may be desirable.

Whether a modification 230 occurs depends on whether and what type of change is observed at 220 to the metric of the patient's autonomic function monitored at 210, as illustrated in several examples above. Modification 230 may not be performed at all, and the method simply returns to 210. If modification occurs, it may to increase or decrease intensity, or to switch programs, before returning to block 210. In some cases, therapy may be stopped, and the method ends at 240.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of“at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient having an implantable medical device system including a lead adapted for placement in the spinal column of a patient and a housing containing each of a sensing circuitry configured to sense a metric of the patient's autonomic function, and a stimulation circuitry configured to issue pulses via the lead; the method comprising:
issuing pulses to the patient in accordance with a first treatment program;
determining, from the sensing circuitry, whether the metric of the patient's autonomic function indicates a risk of overregulation; and
in response to a determination that the metric of the patient's autonomic function indicates a risk of overregulation, reducing the risk of overregulation.

2. The method of claim 1, wherein the step of determining, from the sensing circuitry, whether the metric of the patient's autonomic function indicates a risk of overregulation, is performed by tracking a trend the metric.

3. The method of claim 1, wherein the step of determining, from the sensing circuitry, whether the metric of the patient's autonomic function indicates a risk of overregulation, is performed by:
detecting, using the sensing circuitry, heart beats of the patient;
determining, from the detected heart beats, a cardiac rate of the patient; and
comparing the cardiac rate or a trend of the cardiac rate to a threshold.

4. The method of claim 1, wherein the step of determining, from the sensing circuitry, whether the metric of the patient's autonomic function indicates a risk of overregulation, is performed by:
detecting, using the sensing circuitry, heart beats of the patient;
determining, from the detected heart beats, a heart rate variability of the patient; and
comparing the heart rate variability or a trend of the heart rate variability to a threshold.

5. The method of claim 1, wherein the step of determining, from the sensing circuitry, whether the metric of the patient's autonomic function indicates a risk of overregulation, is performed by:
detecting, using the sensing circuitry, a respiration rate of the patient;
comparing the respiration rate, or a trend of the respiration rate, to a threshold.

6. The method of claim 1, wherein the step of determining, from the sensing circuitry, whether the metric of the patient's autonomic function indicates a risk of overregulation, is performed by:
detecting, using the sensing circuitry, heart beats of the patient;
analyzing the heart beats of the patient to yield a first estimate of the patient's autonomic state;
obtaining data from a second device to yield a second estimate of the patient's autonomic state, wherein the second device provides an indication of blood pressure.

7. The method of claim 1, wherein the step of determining, from the sensing circuitry, whether the metric of the patient's autonomic function indicates a risk of overregulation, is performed by:
detecting, using the sensing circuitry, heart beats of the patient;
analyzing the heart beats of the patient to yield a first estimate of the patient's autonomic state;
obtaining data from a second device to yield a second estimate of the patient's autonomic state, wherein the second device provides an indication of galvanic skin resistance of the patient.

8. The method of claim 1, wherein the step of determining, from the sensing circuitry, whether the metric of the patient's autonomic function indicates a risk of overregulation, is performed by:
determining a first autonomic function marker and a second autonomic function marker each selected from heart rate, heart rate variability, blood pressure, respiration rate, galvanic skin response, bladder, bowel or sexual function, posture and myopotentials; and
using the first autonomic function marker and the second autonomic function marker to estimate the metric.

9. The method of claim 1, wherein the step of determining, from the sensing circuitry, whether the metric of the patient's autonomic function indicates a risk of overregulation, is performed by:
identifying a first marker of autonomic function of the patient,
obtaining an activity signal from the patient indicating a level of intentional activity by the patient, and
determining an effect on the first marker caused by intentional activity of the patient.

10. The method of claim 9, wherein the step of obtaining an activity signal is performed by using an accelerometer in the sensing circuitry.

11. The method of claim 9, wherein the step of obtaining an activity signal is performed by monitoring a component of the electrospinogram.

12. The method of claim 1, wherein the step of reducing the risk of overregulation is performed by reducing one or more of an intensity, a repetition rate, a pulse width, or an amplitude of the pulses delivered to the patient.

13. The method of claim 1, wherein the step of reducing the risk of overregulation is performed by ceasing to issue pulses to the patient in accordance with the first treatment program.

14. The method of claim 13, further comprising, after ceasing to issue pulses to the patient in accordance with the first treatment program, issuing pulses to the patient in accordance with a second treatment program.

15. The method of claim 1, wherein the implantable medical device system further includes a patient remote control external of the patient having a user interface and communication circuitry for communicating with the pulse generator, wherein the step of reducing the risk of overregulation is performed by communicating to the remote control to present a query to the patient regarding stimulation the patient is receiving.

16. The method of claim 1, wherein the step of reducing the risk of overregulation is performed by issuing a communication with an alert that the patient is at risk of overregulation.

17. A method of treating a patient using a spinal cord stimulator, comprising:
generating a mapping of autonomic status of the patient using a first metric and a second metric, the mapping including a first region for a stressed status, and a second region for a relaxed status;
initiating therapy using a first therapy program;
confirming, by tracking the first metric and the second metric, that the patient's autonomic status has shifted from the first region to the second region in response to initiating therapy using the first therapy program;
monitoring the patient's autonomic status in the second region as therapy continues using the first therapy program;
determining that the patient's autonomic status has left the second region;
in response to the determining that the patient's autonomic status has left the second region, determining that the patient is overregulated.

18. The method of claim 17, further comprising, in response to determining that the patient is overregulated, reducing one or more of an intensity, a repetition rate, a pulse width, or an amplitude of pulses delivered to the patient in the first therapy program.

19. The method of claim 17, further comprising, in response to determining that the patient is overregulated, terminating therapy using the first therapy program, and initiating therapy using a second therapy program.

20. The method of claim 17, wherein the first metric is heart rate, and the second metric is heart rate variability.
